(19) United States
(12) Patent Application Publication
(10) Pub. No.: US 2017/0049950 A1
(43) Pub. Date: Feb. 23, 2017

(54) Method for slowing the aging process

(71) Applicant: Marv Enterprises, LLC, Hermitage, PA (US)

(72) Inventor: Mitchell S. Felder, Hermiage, PA (US)

(21) Appl. No.: 15/305,338

(22) PCT Filed: Apr. 14, 2015
(86) PCT No.: PCT/US2015/025649

§ 371 Date: Oct. 19, 2016

Related U.S. Application Data
(60) Provisional application No. 61/988,935, filed on May 6, 2014.

(51) Int. Cl. A61M 1/36; A61M 1/34

(52) U.S. Cl. CPC A61M 1/3687; A61M 1/34; A61M 1/3679; A61M 2205/7545

(57) ABSTRACT

A method of slowing the aging process by extracorporeally treating a patient's blood is described. Certain antigens are known to be associated with aging. The method targets these antigens by complexing them with chemicals that facilitate removal of the antigens from the blood. The blood is removed from the patient, treated with a complexing agent, and then removed from the extracorporeal blood before the blood is returned to the patient. Targeted antigens can include mTOR (mammalian target of rapamycin), insulin growth factor-1 (IGF-1, insulin-like growth factor-1, somatomedin C), lipofuscin, p16 (p16INK4a, CDKN2A, cyclin-dependent kinase inhibitor 2A), SA-beta-gal (senescence-associated beta-gal), PML (promyelocytic leukemia) protein, TGF-β (transforming growth factor-beta), interleukin-6 (IL-6), indoleamine 2,3-dioxygenase, sTNF-R55 (soluble tumor necrosis factor-receptor 55), sTNF-R75 (soluble tumor necrosis factor-receptor 75), progerin, oxygen free radicals (e.g., superoxide, nitric oxide, hydroxyl radical, peroxynitrite, nitrosoperoxycarbonate, hydrogen peroxide, hypochlorite), malondialdehyde (MDA, propanedial), tumor necrosis factor-alpha (TNF-a), and mitogen-activated protein kinases (MAPKs).

### CROSS-REFERENCE TO RELATED APPLICATIONS

This application claims benefit under 35 U.S.C. §119(e) of U.S. Patent Application No. 61/988,935, filed May 6, 2014, which is hereby incorporated herein by reference in its entirety.

### FIELD OF THE INVENTION

The present invention relates to a treatment for slowing the aging process using an extracorporeal methodology.

### BACKGROUND OF THE INVENTION

The aging process can be a major contributor to almost all known diseases. The aging process is especially intrinsic in the development of cancer, strokes, and neurodegenerative diseases. Slowing the aging process would be extremely useful in decreasing morbidity and mortality.

### SUMMARY OF THE INVENTION

The present invention relates to an article and method for extracorporeally treating a patient's blood. The treatment includes a plurality of stages, comprising removing a bodily fluid or blood, hereinafter “blood,” from a patient, applying an extracorporeal treatment to the blood, and returning the blood to the patient.

In the first stage of the treatment, the blood is removed from the patient. A convenient method for removing the blood is using standard venipuncture techniques. In the second stage, a treatment is applied to the blood. The treatment can include an antibody directed against a targeted antigen. The third stage comprises returning the blood to the patient and can also include removing the treatment from the blood before returning the blood to the patient.

### BRIEF DESCRIPTION OF THE DRAWINGS

FIG. 1 is a partial cross sectional view of a cylinder and tubing used to deliver a treatment to a bodily fluid.

FIG. 2 is a partial cross sectional view showing additional detail of the cylinder and tubing of FIG. 1.

### DETAILED DESCRIPTION OF THE INVENTION

The method of the present invention comprises treating a patient's blood extracorporeally with antibody(s) designed to react with particular targeted antigen (TA), including, but not limited to, mTOR (mammalian target of rapamycin), insulin growth factor-1 (IGF-1, insulin-like growth factor-1, somatomedin C), lipofuscin, p16 (p16INK4a, CDKN2A, cyclin-dependent kinase inhibitor 2A), SA-beta-gal (senescence-associated beta-gal), PML (promyelocytic leukemia) protein, TGF-β (transforming growth factor-beta), interleukin-6 (IL-6), indoleamine 2,3-dioxygenase, sTNF-R55 (soluble tumor necrosis factor-receptor 55), sTNF-R75 (soluble tumor necrosis factor-receptor 75), progerin, an oxygen-containing free radical (e.g., superoxide, nitric oxide, hydroxyl radical, peroxynitrite, nitrosoperoxycarbonate, hydrogen peroxide, hypochlorite), malondialdehyde (MDA, propanedial), tumor necrosis factor-α (TNF-alpha), and mitogen-activated protein kinases (MAPKs). The antibody can include a moiety, for example, an albumin moiety, that can complex with the target antigen/TA and thereby permit efficacious dialysis of the antibody-antigen complex. Dialysis methods are well-known by those of skill in the art.

In an embodiment of the invention, the antibody comprises an albumin moiety and targets the removal of the TA from the blood, or body fluid.

The target antigen can be differentiated using standard enzyme-linked immunosorbant assay (ELISA) methodology. ELISA is a biochemical technique that allows the detection of an antigen in a sample. In ELISA, an antigen is affixed to a surface, and then an antibody is used for binding to the antigen. The antibody is linked to an enzyme that enables a color change in the substrate. Other strategies may be employed to validate the level of target antigen(s)/TA(s) in the body fluid, such as, but not limited to, Western blotting technology, UV/vis spectroscopy, mass spectrometry, and surface plasmon resonance (SPR).

An alternative methodology of the present intervention would use a designer antibody with an attached macromolecular moiety instead of an albumin moiety. The macromolecular moiety, attached to the antibody, would be 1.000 mm to 0.00001 mm in diameter. The antibody-macromolecular moiety-targeted antigen complex would then be blocked from reentering the patient's blood, using a series of microscreens which contain openings with a diameter 50% to 99.99999% less than the diameter of the designer antibody-macromolecular moiety. The microscreen opening(s) must have a diameter of at least 25 μm to permit the passage and return to the circulation of the non-pathological blood constituents.

Alternatively, the target antigen(s)/TA(s) can be captured using antibody microarrays, that contain antibodies to the target antigen(s). The antibody microarrays comprise millions of identical monoclonal antibodies attached at high density on glass or plastic slides. After sufficient extracorporeal exposure of the TA(s) to the antibody microarrays, the antibody microarrays-TA(s) may be disposed of, using standard medical practice.

Another alternative methodology of the present intervention comprises removing the targeted antigen(s)/TA(s) from the blood using a designer antibody containing an iron (Fe) moiety. This will then create a Fe-antibody-antigen complex. This iron-containing complex can then be efficaciously removed using a strong, localized magnetic force field.

Alternatively, immunoaffinity chromatography may be used in which the heterogeneous group of molecules in the body fluid will undergo a purification process. There may be entrapment on a solid or stationary phase or medium. Only the targeted antigens (TAs) will be trapped using immunoaffinity chromatography. A solid medium can be removed from the mixture, washed, and the TA(s) may then be released from the entrapment through elution.

Alternatively, gel filtration chromatography may be used in which the body fluid is used to transport the sample through a size-exclusion column that is used to separate the target antigen(s)/TA(s) by size and molecular weight.

Another alternative methodology of the present intervention would use molecular weight cut-off filtration. Molecular weight cut-off filtration refers to the molecular weight at which at least 80% of the target antigen(s)/TA(s) is prohibited from membrane diffusion.

The invention comprises at least three stages, including a first stage, a second stage, and a third stage. The first stage comprises removing blood from a patient. The second stage treats the blood. The thirds stage returns the treated blood to the patient after having achieved the physical removal of the targeted antigen, which can include mTOR (mammalian target of rapamycin), insulin growth factor-1 (IGF-1, insulin-like growth factor-1, somatomedin C), lipofuscin, p16 (p16INK4a, CDKN2A, cyclin-dependent kinase inhibitor 2A), SA-beta-gal (senescence-associated beta-gal), PML (promyelocytic leukemia) protein, TGF-beta (transforming growth factor-beta), interleukin-6 (IL-6), indoleamine 2,3-dioxygenase, sTNF-R55 (soluble tumor necrosis factor-receptor 55), sTNF-R75 (soluble tumor necrosis factor-receptor 75), progerin, an oxygen-containing free radical (e.g., superoxide, nitric oxide, hydroxyl radical, peroxynitrite, nitrosoperoxycarbonate, hydrogen peroxide, hypochlorite), malondialdehyde (MDA, propanedial), tumor necrosis factor-alpha (TNF-α), and mitogen-activated protein kinases (MAPKs), and combinations thereof.

The treatment can include the removal of the targeted antigen(s). The cleansed blood can then be returned to the patient, such as, for example, using the same catheter that was originally used in removing the blood. In one embodiment, the treatment of blood comprises removing 25 mL to 500 mL of blood from a patient, and then applying the treatment to the blood before returning it to the patient. The frequency of such treatments would depend upon an analysis of the underlying symptomatology and pathology of the patient.

The article of the invention includes two stages. The first stage includes an inlet for blood and at least one exterior wall, defining a treatment chamber that is fluidly connected to a second stage. The second stage comprises a removal module and an outlet for the blood. In embodiments, the removal module is selected from the group comprising a mechanical filter, a chemical filter, a dialysis machine, a molecular filter, molecular adsorbant recirculating system (MARS), a plasmapharesis unit, and combinations thereof.

The method includes removing blood from a patient in a first stage, treating the blood to obtain a reduction in the target antigen(s), and optionally removing the treatment from the blood in a second stage, and returning the blood to the patient in a third stage. The blood can be removed from the patient using any convenient method, including standard venipuncture procedure. The second stage can include sequentially passing the extracorporeal blood through a treatment chamber and a removal module.

The second stage applies a treatment to the blood, which can include introducing a designer antibody that joins with a targeted antigen (TA) in the blood to form an antibody-antigen complex. The antibody-antigen complex can be removed from the blood in the removal module. Optionally, the antibody-antigen complex can be conjugated with a second antibody comprising a moiety that increases the efficacy of removal to form an antibody-moiety-antigen complex.

In a third stage, the purified blood, that is, blood that has been cleansed of the TA, can be returned to the patient.

The article of the invention can include two stages. The first stage includes an inlet for the blood and at least one exterior wall defining a treatment chamber that is fluidly connected to a second stage. The second stage comprises a removal module and an outlet for the blood. In embodiments, the removal module can include, for example, a mechanical filter, a chemical filter, a dialysis machine, a magnet, a molecular filter, molecular adsorbant recirculating system (MARS), a plasmapharesis unit, and combinations thereof.

An article for performing the method of the invention comprises a first stage including an inlet for blood and at least one exterior wall defining a treatment chamber that is fluidly connected to a second stage comprising a removal module and an outlet for the blood. The treatment chamber can include a delivery tube for introducing a treatment into the treatment chamber. In embodiments, the delivery tube comprises a hollow tube including at least one interior wall defining a plurality of holes through which the treatment can be added to the treatment chamber. The treatment can also be delivered through the hollow tube in counter-current mode with reference to the flow of the extracorporeal blood. The removal module can be any device capable of removing the antibody-antigen complex.

In an example, the first stage of the device applies a treatment of an antibody with an attached albumin moiety that targets the antigen The second stage includes substantial removal of the treatment from the extracorporeal blood.

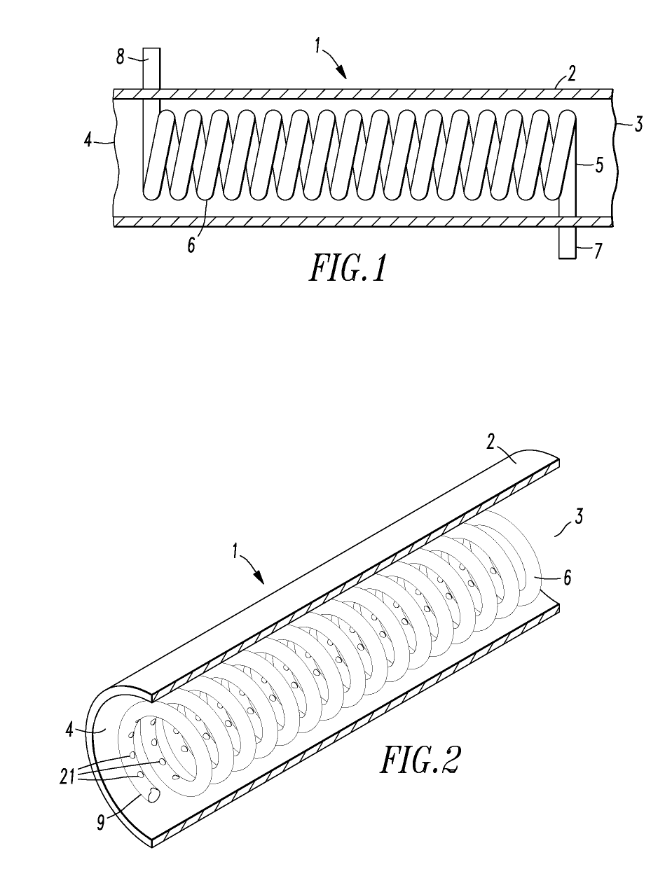

As shown in FIG. 1, the first stage can include an exterior wall 2 defining a treatment chamber 5. The treatment can be applied in the treatment chamber 5. Residence times of the blood can be altered by changing the dimensions of the treatment chamber or the flow rate of the blood through the treatment chamber 5. Blood enters the inlet 3, passes through the treatment chamber 5, and exits the outlet 4. In embodiments, the treatment can be applied from a delivery tube 6 located within the treatment chamber 5. An interior wall 9 defines the delivery tube 6. The delivery tube 6 can include at least one lead 7, 8. The lead 7, 8 can deliver the treatment to the treatment chamber 5. Conveniently, the delivery tubes 6 will have a high contact surface area with the blood. As shown, the delivery tube 6 comprises a helical coil.

With reference to FIG. 2, when the treatment includes the administration of a designer antibody, the delivery tube 6 can be hollow and the interior wall 9 can define a plurality of holes 21. The designer antibodies can be pumped through the delivery tube 6 to effect a desired concentration of designer antibodies in the blood. The designer antibodies can perfuse through the holes 21. The delivery tube 6 can include any suitable material including, for example, metal, plastic, ceramic or combinations thereof. The delivery tube 6 can also be rigid or flexible. In one embodiment, the delivery tube 6 is a metal tube perforated with a plurality of holes. Alternatively, the delivery tube 6 can be plastic.

The antibody with attached albumin moiety, targeting the antigen, can be delivered in a concurrent or counter-current mode with reference to the blood. In counter-current mode, the blood enters the treatment chamber 5 at the inlet 3. The designer antibody can enter through a first lead 8 near the outlet 4 of the treatment chamber 5. Blood then passes to the outlet 4 and the designer antibodies pass to the second lead 7 near the inlet 3. The removal module of the second stage substantially removes the designer antibodies-antigen molecular compound from the blood.

The second stage can include a filter, such as a dialysis machine, which is known to one skilled in the art. The second stage can include a molecular filter, for example, a molecular adsorbents recirculating system (MARS), which may be compatible and/or synergistic with dialysis equipment. MARS technology can be used to remove small-to-average-sized molecules from the blood. Artificial liver filtration presently uses this technique.

The methodology can include a plurality of steps for removing the targeted antigen. A first step can include directing a first antibody against the targeted antigen. A second step can include a second antibody. The second antibody can be conjugated with albumin, or alternatively a moiety that allows for efficacious dialysis. The second antibody or antibody-albumen complex combines with the first antibody forming an antibody-antibody-moiety complex. A third step is then used to remove the complex from the blood. This removal is enabled using dialysis and/or MARS. The purified blood can then be returned to the patient.

In practice, a portion of the purified blood can be tested to ensure a sufficient portion of the targeted antigen has been successfully removed from the blood. Testing can determine the length of treatment and evaluate the efficacy of the sequential dialysis methodology in removing the targeted antigens. Blood with an unacceptably large concentration of complex remaining can then be re-filtered before returning the blood to the patient.

In embodiments, the second stage can remove the antibody-moiety-targeted antigen complex by techniques including, for example, filtering based on molecular size, protein binding, solubility, chemical reactivity, and combinations thereof. For example, a filter can include a molecular sieve, such as zeolite, or porous membranes that capture complexes comprising molecules above a certain size. Membranes can comprise polyacrylonitrile, polysulfone, polyamides, cellulose, cellulose acetate, polyacrylates, polymethylmethacrylates, and combinations thereof. Increasing the flow rate or diasylate flow rate can increase the rate of removal of the antibody with an attached albumin moiety targeting the antigen.

Additional embodiments can include continuous renal replacement therapy (CRRT), which can remove large quantities of filterable molecules from the extracorporeal body fluid. CRRT would be particularly useful for molecular compounds that are not strongly bound to plasma proteins. Categories of CRRT include continuous arteriovenous hemofiltration, continuous venovenous hemofiltration, continuous arteriovenous hemodiafiltration, slow continuous filtration, continuous arteriovenous high-flux hemodialysis, and continuous venovenous high flux hemodialysis.

The sieving coefficient (SC) is the ratio of the molecular concentration in the filtrate to the incoming blood. A SC close to zero indicates that the moiety antibody-targeted antigen complex will not pass through the filter. A filtration rate of about 10 mL per min is generally satisfactory. Other methods of increasing the removability of the moiety-antibody-targeted antigen include the use of temporary acidification of the blood using organic acids to compete with protein binding sites.

Embodiments of the present invention include a method for slowing aging comprising:
a. removing a body fluid from a patient;
b. applying a treatment to the body fluid that targets an antigen associated with aging; and
c. returning the body fluid to the patient in a third stage.

Embodiments of the present invention also include such a method wherein the method includes removing the treatment from the body fluid.

Embodiments of the present invention also include such a method wherein the antigen is selected from a group consisting of mTOR, insulin growth factor-1, lipofuscin, p16, SA-beta-gal, PML protein, TGF-beta, interleukin-6, indoleamine-2,3-dioxygenase, sTNF-R55, sTNF-R75, progerin, an oxygen-containing free radical, malondialdehyde, tumor necrosis factor-alpha, mitogen-activated protein kinase, and combinations thereof.

Embodiments of the present invention also include such a method wherein the treatment includes
a. introducing an antibody that joins with the antigen to form an antibody-antigen complex; and
b. removing the complex from the body fluid.

Embodiments of the present invention also include such a method wherein the treatment includes
a. introducing a targeted antibody that combines with the antigen to form an antibody-antigen complex; and
b. conjugating the antibody-antigen complex with a second antibody comprising a moiety that increases the efficacy of removal to form an antibody-moiety-antigen complex.

Embodiments of the present invention also include such a method wherein the method includes testing the body fluid after the treatment and before returning the body fluid to the patient in order to determine efficacy.

Unless otherwise indicated, all numbers expressing quantities of ingredients, properties such as molecular weight, reaction conditions, and so forth used in the specification and claims are to be understood as being modified in all instances by the term “about.”

Accordingly, unless indicated to the contrary, the numerical parameters set forth in the following specification and attached claims are approximations that may vary depending upon the desired properties sought to be obtained by the present invention.

At the very least, and not as an attempt to limit the application of the doctrine of equivalents to the scope of the claims, each numerical parameter should at least be construed in light of the number of reported significant digits and by applying ordinary rounding techniques.

While the invention has been particularly shown and described with reference to a preferred embodiment, it will be understood by those skilled in the art that various changes in form and detail may be made therein without departing from the spirit and scope of the invention. All documents, books, manuals, papers, patents, published patent applications, guides, abstracts, and other references cited herein are incorporated by reference in their entirety.

Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

1. A method for slowing aging comprising:
a. removing a body fluid from a patient;
b. applying a treatment to the body fluid that targets an antigen associated with aging; and
c. returning the body fluid to the patient in a third stage.

2. The method of claim 1, wherein the method includes removing the treatment from the body fluid.

3. The method of claim 1, wherein the antigen is selected from the group consisting of mTOR, insulin growth factor-1, lipofuscin, p16, SA-beta-gal, PML protein, TGF-beta, interleukin-6, indoleamine 2,3-dioxygenase, sTNF-R55, sTNF-R75, progerin, an oxygen-containing free radical, malondialdehyde, tumor necrosis factor-alpha, mitogen-activated protein kinases, and combinations thereof.

4. The method of claim 1, wherein the treatment includes
a. introducing an antibody that joins with the antigen to form an antibody-antigen complex; and
b. removing the complex from the body fluid.

5. The method of claim 1, wherein the treatment includes
a. introducing a targeted antibody that combines with the antigen to form an antibody-antigen complex; and
b. conjugating the antibody-antigen complex with a second antibody comprising a moiety that increases the efficacy of removal to form an antibody-moiety-antigen complex.

6. The method of claim 1, wherein the method includes testing the body fluid after the treatment and before returning the body fluid to the patient in order to determine efficacy.
